(19) United States
(12) Patent Application Publication
(10) Pub. No.: US 2005/0107405 A1
(43) Pub. Date: May 19, 2005

(54) Pharmaceutical composition for alleviating pain or spasticity in a patient suffering from spinal cord injury

(76) Inventor: Satoshi Takasaka, Tokyo (JP)

Correspondence Address: WARNER-LAMBERT COMPANY, 2800 PLYMOUTH RD, ANN ARBOR, MI, 48105, US

(21) Appl. No.: 10/787,470

(22) Filed: Feb. 26, 2004

(30) Foreign Application Priority Data
Feb. 28, 2003 (JP) 2003-053884

(51) Int. Cl. A61K 31/519

(52) U.S. Cl. 514/262.1

(57) ABSTRACT

To provide a novel pharmaceutical composition for alleviating pain or spasticity in a patient suffering from spinal cord injury. The present invention relates to a method for alleviating pain or spasticity in a patient suffering from spinal cord injury comprising administering to the patient an effective amount of cGMP PDE5 inhibitor, to a pharmaceutical composition comprising such an effective amount of the inhibitor, and to a use of the inhibitor in the manufacture of such a pharmaceutical composition.

This application is a United States utility application, which claims the benefit of priority to Japanese Application Serial No. JP2003-053884 filed Feb. 28, 2003.

### TECHNICAL FIELD

The present invention relates to a method for alleviating pain or spasticity in a patient suffering from spinal cord injury comprising the step of administering to the patient a cGMP PDE5 (cyclic guanosine monophosphate phosphodiesterase type five) inhibitor, a pharmaceutical composition for alleviating pain or spasticity in a patient suffering from spinal cord injury, comprising an effective amount of a cGMP PDE5 inhibitor, and a use of a cGMP PDE5 inhibitor in the manufacture of the pharmaceutical composition.

### BACKGROUND ART

WO94/28902 discloses a cGMP PDE5 inhibitor is effective as a medicament for the curative treatment of Male Erectile Dysfunction (MED) (see Patent Reference No. 1). Based on this finding, a compound having the common name sildenafil citrate, the chemical name 1-[[3-(6,7-dihydro-1-methyl-7-oxo-3-propyl-1H-pyrazolo[4,3-d]pyrimidin-5-yl)-4-ethoxyphenyl]sulfonyl]-4-methylpiperazine monocitrate, and the trade name VIAGRA® has been developed and in remarkable success as a medicament for the curative treatment of MED.

Spinal cord injury is defined as an injury of spine or spinal cord, and depending on its nature, is classified in open injury (caused by puncture wound, gunshot wound) and closed injury. Most of spinal cord injury are closed injury. In most cases, spinal cord is injured by spine fracture or by a force associated with dislocation. It, however, is sometime injured by a simple hyperflexion or hyperextension of spinal cord. In many cases, spinal cord injury is found in the cervical vertebrae-thoracic vertebrae transition and the thoracic vertebrae-lumber vertebrae transition. Spinal cord injury is traditionally classified in spinal cord concussion, spinal cord contusion, rupture, and spinal hemangioma (see Current Medical Dictionary, Ishiyaku-shuppan Kabushiki-Kaisha). [Spinal cord contusion means a structural injury of spinal cord. Complete separation of dura mater from spinal cord is considered the highest level of the primary injury, however in most cases, no rupture is found in dura mater. Depending on the severity of the secondary injury, such as hemorrhage from spinal cord, edema, developed after the primary injury, kinesthesia paralysis, visicorectal disorder, or autonomic disorder below the level of the injured site takes place, which is under the condition of incomplete separation (see Current Medical Dictionary, Ishiyaku-shuppan Kabushiki-Kaisha).

The severity of pain or spasticity caused by spinal cord injury normally increases in days a at lower temperature or in low atmospheric pressure, and in such situation, the pain is keen and hard to keep self-controlled.

Japanese Unexamined Patent Publication (Kokai) No.2001-122803 discloses that cGMP PDE5 inhibitors including sildenafil are effective in the treatment of neuropathy, in particular diabetic neuropathy (see Patent Reference No. 2).

N. K. Jain et al., Brain Research 909 (2001) 170-178 describes that sildenafil induces antinociception in the peripheral nociception, and the effect of analgesia could be potentiated by sodium nitroprasside and L-arginine, probably through the activation of the NO-cGMP pathway (see Non-Patent Reference No. 1).

R. Asomoza-Espinosa et al., European Journal of Pharmacology 418 (2001) 195-2000 describes that sildenafil produces antinociceptive activity, and increases that of diclofenac, probably through the inhibition of cGMP degradation (see Non-Patent Reference No. 2).

T. Mixcoatl-Zecuatl et al., European Journal of Pharmacology 400 (2000) 81-87 describes that sildenafil significantly increases the morphine-induced antinociception, and increases the morphine-induced antinociception, probably through the inhibition of cGMP degradation (see Non-Patent Reference No. 3).

[Patent Reference No. 1]

WO 94/28902

[Patent Reference No. 2]

Japanese Unexamined Patent Publication (Kokai) No. 2001-122803

[Non-Patent Reference No. 1]

N. K. Jain et al., Bain Research 909 (2001) 170-178

[Non-Patent Reference No. 2]

R. Asomoza-Espinosa et al., European Journal of Pharmacology 418 (2001) 195-200

[Non-Patent Reference No. 3]

T. Mixcoatl-Zecuatl et al., European Journal of Pharmacology 400 (2000) 81-87

### DETAILED DESCRIPTION OF THE INVENTION

It has been now surprisingly found that an administration of sildenafil citrate to a patient suffering from spinal cord injury for the purpose of improving or treating the sexual dysfunction of the patient, unexpectedly alleviates a pain or spasticity in the patient. The severity of pain or spasticity caused by spinal cord injury normally increases in days at lower temperature or in low atmospheric pressure, and in such a situation, the pain is keen and hard to keep self-controlled.

It is totally unexpected that an administration of sildenafil citrate significantly alleviates the pain or spasticity caused by spinal cord injury, because an administration of analgesics, such as Loxonin, Voltaren (diclofenac) for the same purpose does not have any analgesic effects on alleviating the pain caused by spinal cord injury. Further, the effect of sildenafil citrate is remarkable.

In view of the above situation, it is obvious that there is a need for providing a novel medicament which can effectively alleviate the pain or spasticity caused by spinal cord injury.

Sildenafil citrate belongs to a class of cGMP PDE5 inhibitors, and inherently possesses vasodilation activity.

Without wishing to be bound on any particular theory, it is considered that one of the causes of the pain or spasticity caused by spinal cord injury is contraction or hemodynamics disorder of the peripheral vascularity caused by spinal cord injury. Thus, it is also considered that the vasodilation activity or any other pharmacological activities of sildenafil citrate alleviate the hemodynamics disorder of the peripheral vascularity, which eventually alleviates the pain or spasticity.

It should, however, be noted that a true mechanism in which the pharmaceutical composition of the present invention can exert such a pain or spasticity-alleviating effect, has not yet been known.

In one embodiment of the present invention, there is provided a pharmaceutical composition for alleviating pain or spasticity in patient suffering from spinal cord injury, comprising an effective amount of a cGMP PDE5 inhibitor.

The inhibitor may be administered orally.

The daily dosage may be 5 to 500 mg, the inhibitor may have an IC₅₀ at less than 100 nanomolar, and may have a selectivity ratio in excess of 100.

The inhibitor may be a compound of formula(I): 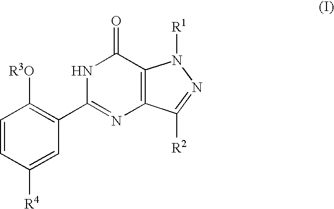
wherein R¹ is H; C₁-C₃ alkyl; C₁-C₃ perfluoroalkyl; or C₃-C₅ cycloalkyl;
R² is H; C₁-C₆ alkyl optionally substituted with C₃-C₆ cycloalkyl; C₁-C₃ perfluoroalkyl; or C₃-C₆ cycloalkyl;
R³ is C₁-C₆ alkyl optionally substituted with C₃-C₆ cycloalkyl; C₁-C₆ perfluoroalkyl; C₃-C₅ cycloalkyl; C₃-C₆ alkenyl; or C₃-C₆ alkynyl;
R⁴ is C₁-C₄ alkyl optionally substituted with OH, NR⁵R⁶, CN, CONR⁵R⁶ or CO₂R⁷; C₂-C₄ alkenyl optionally substituted with CN, CONR⁵R⁶ or CO₂R⁷; C₂-C₄ alkanoyl optionally substituted with NR⁵R⁶; (hydroxy)C₂-C₄ alkyl optionally substituted with NR⁵ R⁶; (C₂-C₃ alkoxy)C₁-C₂ alkyl optionally substituted with OH or NR⁵R⁶; CONR⁵R⁶, CO₂R⁷; halo; NR⁵R⁶; NHSO₂NR⁵R⁶; NHSO₂R⁸; SO₂NR⁹R¹⁰; or phenyl, pyridyl, pyrimidinyl, imidazolyl, oxazolyl, thiazolyl, thienyl or triazolyl any of which is optionally substituted with methyl;
R⁵ and R⁶ are each independently H or C₁-C₄ alkyl, or together with the nitrogen atom to which they are attached form a pyrrolidinyl, piperidino, morpholino, 4-N(R¹¹)-piperazinyl or imidazolyl group wherein said group is optionally substituted with methyl or OH;
R⁷ is H or C₁-C₄ alkyl;
R⁸ is C₁-C₃ alkyl optionally substituted with NR⁵R⁶;
R⁹ and R¹⁰ together with the nitrogen atom to which they are attached form a pyrrolidinyl, piperidino, morpholino or 4-N(R¹²)-piperazinyl group wherein said group is optionally substituted with C₁-C₄ alkyl, C₁-C₃ alkoxy, NR¹³R¹⁴ or CONR¹³R¹⁴;
R¹¹ is H; C₁-C₃ alkyl optionally substituted with phenyl; (hydroxy)C₂-C₃ alkyl; or C₁-C₄ alkanoyl;
R¹² is H; C₁-C₆ alkyl; (C₁-C₃ alkoxy)C₂-C₆ alkyl; (hydroxy)C₂-C₆ alkyl; (R¹³R¹⁴N)C₂-C₆ alkyl; (R¹³R¹⁴NOC)C₁-C₆ alkyl; CONR¹³R¹⁴; CSNR¹³R¹⁴; or C(NH)NR¹³R¹⁴;
and R¹³ and R¹⁴ are each independently H; C₁-C₄ alkyl; (C₁-C₃ alkoxy)C₂-C₄ alkyl; or (hydroxy)C₂-C₄ alkyl;
   or a pharmaceutically acceptable salt thereof.

The inhibitor may also be sildenafil, or pharmaceutically acceptable salts thereof, and the daily dosage may be 10 to 100 mg.

In another embodiment of the present invention, there is provided a use of a cGMP-PDE5 inhibitor in the manufacture of a medicament for alleviating pain or spasticity in a patient suffering from spinal cord injury. The inhibitor may be administered orally.

The daily dosage may be 5 to 500 mg, the inhibitor may have an IC₅₀ at less than 100 nanomolar, and may have a selectivity ratio in excess of 100.

The inhibitor may be a compound of formula(I): 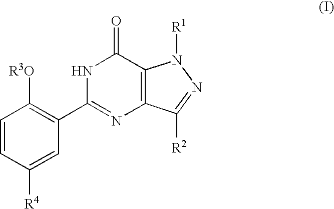
wherein R¹ is H; C₁-C₅ cycloalkyl;
R² is H; C₁-C₆ cycloalkyl; C₁-C₃ perfluoroalkyl; or C₃-C₆ cyc
R³ is C₁-C₆ alkyl optionally substituted with C₃-C₆ cycloalkyl; C₁-C₆ perfluoroalkyl; C₃-C₅ cycloalkyl; C₃-C₆ alkenyl; or C₃-C₆ alkynyl;
R⁴ is C₁-C₄ alkyl optionally substituted with OH, NR⁵R⁶, CN, CONR⁵R⁶ or CO₂R⁷; C₂-C₄ alkenyl optionally substituted with CN, CONR⁵R⁶ or CO₂R⁷; C₂-C₄ alkanoyl optionally substituted with NR⁵R⁶; (hydroxy)C₂-C₄ alkyl optionally substituted with NR⁵R⁶; (C₂-C₃ alkoxy)C₁-C₂ alkyl optionally substituted with OH or NR⁵R⁶; CONR⁵R⁶; CO₂R⁷; halo; NR⁵R⁶; NHSO₂NR⁵R⁶; NHSO₂R⁸; SO₂NR⁹R¹⁰; or phenyl, pyridyl, pyrimidinyl, imidazolyl, oxazolyl, thiazolyl, thienyl or triazolyl any of which is optionally substituted with methyl;
R⁵ and R⁶ are each independently H or C₁-C₄ alkyl, or together with the nitrogen atom to which they are attached form a pyrrolidinyl, piperidino, morpholino, 4-N(R¹¹)-piperazinyl or imidazolyl group wherein said group is optionally substituted with methyl or OH;
R⁷ is H or C₁-C₄ alkyl;
R⁸ is C₁-C₃ alkyl optionally substituted with NR⁵R⁶;
R⁹ and R¹⁰ together with the nitrogen atom to which they are attached form a pyrrolidinyl, piperidino, morpholino or 4-N(R¹²)-piperazinyl group wherein said group is optionally substituted with C₁-C₄ alkyl, C₁-C₃ alkoxy, NR¹³R¹⁴ or CONR¹³R¹⁴;
R¹¹ is H; C₁-C₃ alkyl optionally substituted with phenyl; (hydroxy)C₂-C₃ alkyl; or C₁-C₄ alkanoyl;
R¹² is H; C₁-C₆ alkyl; (C₁-C₃ alkoxy)C₂-C₆ alkyl; (hydroxy)C₂-C₆ alkyl; (R¹³R¹⁴N)C₂-C₆ alkyl; (R¹³R¹⁴NOC)C₁-C₆ alkyl; CONR¹³R¹⁴; CSNR¹³R¹⁴; or C(NH)NR¹³R¹⁴;
and R¹³ and R¹⁴ are each independently H; C₁-C₄ alkyl; (C₁-C₃ alkoxy)C₂-C₄ alkyl; or (hydroxy)C₂-C₄ alkyl;
   or a pharmaceutically acceptable salt thereof.

The inhibitor may also be sildenafil, or pharmaceutically acceptable salts thereof, and the daily dosage may be 10 to 100 mg.

In another embodiment of the present invention, there is provided a method for alleviating pain or spasticity in a patient suffering from spinal cord injury, comprising the step of administering to the patient such an effective amount of a cGMP PDE5 inhibitor sufficient to alleviate the pain or spasticity. The inhibitor may be administered orally.

The daily dosage of the inhibitor may be 5 to 500 mg, the inhibitor may have an IC₅₀ at less than 100 nanomolar, and may have a selectivity ratio in excess of 100.

The inhibitor may be a compound of formula(I): 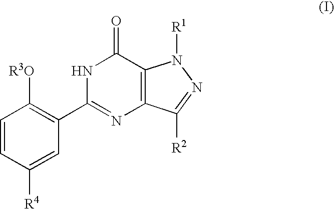
wherein R¹ is H; C₁-C₃ alkyl; C₁-C₃ perfluoroalkyl; or C₃-C₅ cycloalkyl;
R² is H; C₁-C₆ alkyl optionally substituted with C₃-C₆ cycloalkyl; C₁-C₃ perfluoroalkyl; or C₃-C₆ cycloalkyl;
R³ is C₁-C₆ alkyl optionally substituted with C₃-C₆ cycloalkyl; C₁-C₆ perfluoroalkyl; C₃-C₅ cycloalkyl; C₃-C₆ alkenyl; or C₃-C₆ alkynyl;
R⁴ is C₁-C₄ alkyl optionally substituted with OH, NR⁵R⁶, CN, CONR⁵R⁶ or CO₂R⁷; C₂-C₄ alkenyl optionally substituted with CN, CONR⁵R⁶ or CO₂R⁷; C₂-C₄ alkanoyl optionally substituted with NR⁵R⁶; (hydroxy)C₂-C₄ alkyl optionally substituted with NR⁵R⁶; (C₂-C₃ alkoxy)C₁-C₂ alkyl optionally substituted with OH or NR⁵R⁶; CONR⁵R⁶; CO₂R⁷; halo; NR⁵R⁶; NHSO₂NR⁵R⁶; NHSO₂R⁸; SO₂NR⁹R¹⁰; or phenyl, pyridyl, pyrimidinyl, imidazolyl, oxazolyl, thiazolyl, thienyl or triazolyl any of which is optionally substituted with methyl;
R⁵ and R⁶ are each independently H or C₁-C₄ alkyl, or together with the nitrogen atom to which they are attached form a pyrrolidinyl, piperidino, morpholino, 4-N(R¹¹)-piperazinyl or imidazolyl group wherein said group is optionally substituted with methyl or OH;
R⁷ is H or C₁-C₄ alkyl;
R⁸ is C₁-C₃ alkyl optionally substituted with NR⁵R⁶;
R⁹ and R¹⁰ together with the nitrogen atom to which they are attached form a pyrrolidinyl, piperidino, morpholino or 4-N(R¹²)-piperazinyl group wherein said group is optionally substituted with C₁-C₄ alkyl, C₁-C₃ alkoxy, NR¹³R¹⁴ or CONR¹³R¹⁴;
R¹¹ is H; C₁-C₃ alkyl optionally substituted with phenyl; (hydroxy)C₂-C₃ alkyl; or C₁-C₄ alkanoyl;
R¹² is H; C₁-C₆ alkyl; (C₁-C₃ alkoxy)C₂-C₆ alkyl; (hydroxy)C₂-C₆ alkyl; (R¹³R¹⁴N)C₂-C₆ alkyl; (R¹³R¹⁴NOC)C₁-C₆ alkyl; CONR¹³R¹⁴; CSNR¹³R¹⁴; or C(NH)NR¹³R¹⁴;
and R¹³ and R¹⁴ are each independently H; C₁-C₄ alkyl; (C₁-C₃ alkoxy)C₂-C₄ alkyl; or (hydroxy)C₂-C₄ alkyl;
   or a pharmaceutically acceptable salt thereof.

The inhibitor may be sildenafil, or pharmaceutically acceptable salts thereof, and the daily dosage may be 10 to 100 mg.

Suitable cGMP PDE5 inhibitors for the use according to the present invention include:
the pyrazolo [4,3-d]pyrimidin-7-ones disclosed in EP-A-0463756; the pyrazolo [4,3-d]pyrimidin-7-ones disclosed in EP-A-0526004; the pyrazolo [4,3-d]pyrimidin-7-ones disclosed in published international patent application WO93/06104; the isomeric pyrazolo [3,4-d]pyrimidin-4-ones disclosed in published international patent application WO93/07149; the quinazolin-4-ones disclosed in published international patent application WO 93/12095; the pyrido [3,2-d]pyrimidin-4-ones disclosed in published international patent application WO 94/05661; the purin-6-ones disclosed in published international patent application WO 94/00453; the pyrazolo [4,3-d]pyrimidin-7-ones disclosed in published international patent application WO 98/49166; the pyrazolo [4,3-d]pyrimidin-7-ones disclosed in published international patent application WO 99/54333; the pyrazolo [4,3-d]pyrimidin-4-ones disclosed in EP-A-0995751; the pyrazolo [4,3-d]pyrimidin-7-ones disclosed in published international patent application WO00/24745; the pyrazolo [4,3-d]pyrimidin-4-ones disclosed in EPA0995750; the hexahydropyrazino [2′,1′:6,1]pyrido[3,4-b]indole-1,4-diones disclosed in published international application WO95/19978; the imidazo[5,1-f][1,2,4]triazin-ones disclosed in EP-A-1092719 and in published international application WO99/24433 and the bicyclic compounds disclosed in published international application WO93/07124.

Further examples of suitable PDE5 inhibitors for use herein include: the pyrazolo [4,3-d]pyrimidin-7-ones disclosed in published international application WO 01/27112; the pyrazolo [4,3-d]pyrimidin-7-ones disclosed in published international application WO01/27113; the compounds disclosed in EP-A-1092718 and the compounds disclosed in EP-A-1092719; the tricyclic compounds disclosed in EP-A-1241170; the alkyl sulphone compounds disclosed in published international application WO02/074774; the compounds disclosed in published international application WO02/072586; the compounds disclosed in published international application WO 02/079203 and the compounds disclosed in WO02/074312.

Preferred type V phosphodiesterase inhibitors for the use according to the present invention include:
5-[2-ethoxy-5-(4-methyl-1-piperazinylsulphonyl)phenyl]-1-methyl-3-n-propyl-1,6-dihydro-7H-pyrazolo[4,3-d]pyrimidin-7-one (sildenafil) also known as 1-[[3-(6,7-dihydro-1-methyl-7-oxo-3-propyl-1H-pyrazolo[4,3-d]pyrimidin-5-yl)-4-ethoxyphenyl]sulphonyl]-4-methylpiperazine (see EP-A-0463756);
5-(2-ethoxy-5-morpholinoacetylphenyl)-1-methyl-3-n-propyl-1,6-dihydro-7H-pyrazolo[4,3-d]pyrimidin-7-one (see EP-A-0526004);
3-ethyl-5-[5-(4-ethylpiperazin-1-ylsulphonyl)-2-n-propoxyphenyl]-2-(pyridin-2-yl)methyl-2,6-dihydro-7H-pyrazolo[4,3-d]pyrimidin-7-one (see WO98/49166);
3-ethyl-5-[5-(4-ethylpiperazin-1-ylsulphonyl)-2-(2-methoxyethoxy)pyridin-3-yl]-2-(pyridin-2-yl)methyl-2,6-dihydro-7H-pyrazolo[4,3-d]pyrimidin-7-one (see WO99/54333);
(+)-3-ethyl-5-[5-(4-ethylpiperazin-1-ylsulphonyl)-2-(2-methoxy-1(R)-methylethoxy(pyridin-3-yl]-2-methyl-2,6-dihydro-7H-pyrazolo[4,3-d]pyrimidin-7-one, also known as 3-ethyl-5-{5-[4-ethylpiperazin-1-ylsulphonyl]-2-([(1R)-2-methoxy-1-methylethyl]oxy)pyridin-3-yl}-2-methyl-2,6-dihydro-7H-pyrazolo[4,3-d]pyrimidin-7-one (see WO99/54333);
5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one, also known as 1-{6-ethoxy-5-[3-ethyl-6,7-dihydro-2-(2-methoxyethyl)-7-oxo-2H-pyrazolo[4,3-d]pyrimidin-5-yl]-3-pyridylsulphonyl}-4-ethylpiperazine (see WO 01/27113, Example 8);
5-[2-iso-Butoxy-5-(4-ethylpiperazin-1-ylsulphonyl)pyridin-3-yl]-3-ethyl-2-(1-methylpiperidin-4-yl)-2,6-dihydro-7H-pyrazolo[4,3-d]pyrimidin-7-one (see WO 01/27113, Example 15);
5-[2-Ethoxy-5-(4-ethylpiperazin-1-ylsulphonyl)pyridin-3-yl]-3-ethyl-2-phenyl-2,6-dihydro-7H-pyrazolo[4,3-d]pyrimidin-7-one (see WO 01/27113, Example 66);
5-(5-Acetyl-2-propoxy-3-pyridinyl)-3-ethyl-2-(1-isopropyl-3-azetidinyl)-2,6-dihydro-7H-pyrazolo[4,3-d]pyrimidin-7-one (see WO 01/27112, Example 124);
5-(5-Acetyl-2-butoxy-3-pyridinyl)-3-ethyl-2-(1-ethyl-3-azetidinyl)-2,6-dihydro-7H-pyrazolo[4,3-d]pyrimidin-7-one (see WO01/27112, Example 132);
(6R,12aR)-2,3,6,7,12,12a-hexahydro-2-methyl-6-(3,4-methylenedioxyphenyl) pyrazino[2′,1′:6,1]pyrido[3,4-b]indole-1,4-dione (tadalafil, IC-351, Cialis®), i.e. the compound of examples 78 and 95 of published international application WO95/19978, as well as the compound of examples 1, 3, 7 and 8;
2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil) also known as 1-[[3-(3,4-dihydro-5-methyl-4-oxo-7-propylimidazo[5,1-f]-as-triazin-2-yl)-4-ethoxyphenyl]sulphonyl]-4-ethylpiperazine, i.e. the compound of examples 20, 19, 337 and 336 of published international application WO99/24433;
The compound of example 11 of published international application WO93/07124 (EISAI);
Compounds 3 and 14 from Rotella D P, J. Med. Chem., 2000, 43, 1257;
4-(4-chlorobenzyl)amino-6,7,8-trimethoxyquinazoline; and
7,8-dihydro-8-oxo-6-[2-propoxyphenyl]-1H-imidazo[4,5-g]quinazoline and 1-[3-[1-[(4-fluorophenyl)methyl]-7,8-dihydro-8-oxo-1H-imidazo[4,5-g]quinazolin-6-yl]-4-propoxyphenyl]carboxamide.

Still other type cGMP PDE5 inhibitors useful in conjunction with the present invention include: 4-bromo-5-(pyridylmethylamino)-6-[3-(4-chlorophenyl)-propoxy]-3(2H)pyridazinone; 1-[4-[(1,3-benzodioxol-5-ylmethyl)amiono]-6-chloro-2-quinozolinyl]-4-piperidine-carboxylic acid, monosodium salt; (+)-cis-5,6a,7,9,9,9a-hexahydro-2-[4-(trifluoromethyl)-phenylmethyl-5-methyl-cyclopent-4,5]imidazo[2,1-b]purin-4(3H)one; furazlocillin; cis-2-hexyl-5-methyl-3,4,5,6a,7,8,9,9a-octahydrocyclopent[4,5]-imidazo[2,1-b]purin-4-one; 3-acetyl-1-(2-chlorobenzyl)-2-propylindole-6-carboxylate; 3-acetyl-1-(2-chlorobenzyl)-2-propylindole-6-carboxylate; 4-bromo-5-(3-pyridylmethylamino)-6-(3-(4-chlorophenyl) propoxy)-3-(2H)pyridazinone; I-methyl-5(5-morpholinoacetyl-2-n-propoxyphenyl)-3-n-propyl-1,6-dihydro-7H-pyrazolo(4,3-d)pyrimidin-7-one; 1-[4-[(1,3-benzodioxol-5-ylmethyl)amino]-6-chloro-2-quinazolinyl]-4-piperidinecarboxylic acid, monosodium salt; Pharmaprojects No. 4516 (Glaxo Wellcome); Pharmaprojects No. 5051 (Bayer); Pharmaprojects No. 5064 (Kyowa Hakko; see WO 96/26940); Pharmaprojects No. 5069 (Schering Plough); GF-196960 (Glaxo Wellcome); E-8010 and E-4010 (Eisai); Bay-38-3045 & 38-9456 (Bayer); FR229934 and FR226807 (Fujisawa); and Sch-51866.

It is to be understood that the contents of the above published patent applications, and in particular the general formulae and exemplified compounds therein are incorporated herein in their entirety by reference thereto.

The suitability of any particular cGMP PDE5 inhibitor can be readily determined by evaluation of its potency and selectivity using literature methods followed by evaluation of its toxicity, absorption, metabolism, pharmacokinetics, etc in accordance with standard pharmaceutical practice.

Preferably, the cGMP PDE5 inhibitors have an IC₅₀ at less than 100 nanomolar, more preferably, at less than 50 nanomolar, more preferably still at less than 10 nanomolar.

IC₅₀ values for the cGMP PDE5 inhibitors may be determined using established literature methodology, for example as described in EP 0463756-B1 and EP0526004-A1.

Preferably the cGMP PDE5 inhibitors used in the pharmaceutical combinations according to the present invention are selective for the PDE5 enzyme. Preferably they have a selectivity of PDE5 over PDE3 of greater than 100 more preferably greater than 300. More preferably the PDE5 has a selectivity over both PDE3 and PDE4 of greater than 100, more preferably, greater than 300.

Selectivity ratios may readily be determined by the skilled person. IC50 values for the PDE3 and PDE4 enzyme may be determined using established literature methodology, see S A Ballard et al, Journal of Urology, 1998, vol.159, pages 2164-2171 and as detailed herein after.

Surprisingly, the cGMP PDE5 inhibitors, such as sildenafil, can be used to alleviate pain or spasticity in a patient suffering from spinal cord injury, systemically, preferably by mouth.

The cGMP PDE5 inhibitors can be administered alone but, in human therapy will generally be administered in admixture with a suitable pharmaceutical excipient diluent or carrier selected with regard to the intended route of administration and standard pharmaceutical practice.

For example, the cGMP PDE5 inhibitors can be administered orally, buccally or sublingually in the form of tablets, capsules, ovules, elixirs, solutions or suspensions, which may contain flavouring or colouring agents, for im-mediate-, delayed-, modified-, or controlled-release applications.

Such tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethyl cellulose, hydroxypropylcellulose, sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the cGMP PDE5 inhibitors of the invention may be combined with various sweetening or flavouring agents, colouring matter or dyes, with emulsifying and/or suspending agents and with diluents such as water, ethanol, propylene glycol and glycerin, and combinations thereof.

The cGMP PDE5 inhibitors can also be administered parenterally, for example, intravenously, intra-arterially, intraperitoneally, intramuscularly or subcutaneously, or they may be administered by infusion techniques. For such parenteral administration they are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

The dosage of cGMP PDE5 inhibitor in such formulations will depend on its potency, but can be expected to be in the range of from 1 to 500 mg for administration up to three times a day. For oral and parenteral administration to human patients, the daily dosage level of the cGMP PDE5 inhibitor will usually be from 5 to 500 mg (in single or divided doses). In the case of sildenafil, a preferred dose is in the range 10 to 100 mg which can be administered up to three times a day. However the precise dose will be as determined by the prescribing physician and will depend on the age and weight of the patient and severity of the symptoms.

Thus, for example, tablets or capsules of the cGMP PDE5 inhibitor may contain from 5 to 250 mg (e.g. 10 to 100 mg) of active compound for administration singly or two or more at a time, as appropriate. The physician in any event will determine the actual dosage which will be most suitable for any individual patient and it will vary with the age, weight and response of the particular patient. The above dosages are exemplary of the average case. There can, of course, be individual instances where higher or lower dosage ranges are merited and such are within the scope of this invention.

The cGMP PDE5 inhibitors can also be administered intranasally or by inhalation and are conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetrafluoroethane, a hydrofluoroalkane such as 1,1,1,2-tetrafluoroethane or 1,1,1,2,3,3,3-heptafluoropropane, carbon dioxide or other suitable gas. In the case of a pressurised aerosol, the dosage unit may be determined by providing a valve to deliver a metered amount. The pressurised container, pump, spray or nebuliser may contain a solution or suspension of the cGMP PDE5 inhibitor, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of the cGMP PDE5 inhibitor and a suitable powder base such as lactose or starch.

Aerosol or dry powder formulations are preferably arranged so that each metered dose or “puff” contains from 1 to 50 mg of the cGMP PDE5 inhibitor, for delivery to the patient.

The overall daily dose with an aerosol will be in the range of from 1 to 50 mg which may be administered in a single dose or, more usually, in divided doses throughout the day.

Alternatively, the cGMP PDE5 inhibitors can be administered in the form of a suppository or pessary. The cGMP PDE5 inhibitor may be applied topically in the form of a gel, hydrogel, lotion, solution, cream, ointment or dusting powder. The cGMP PDE5 inhibitors may also be dermally or transdermally administered, for example, by the use of a skin patch.

For application topically to the skin, the cGMP PDE5 inhibitors can be formulated as a suitable ointment containing the inhibitor suspended or dissolved in, for example, a mixture with one or more of the following: mineral oil, liquid petrolatum, white petrolatum, propylene glycol, polyoxyethylene polyoxypropylene compound, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

The cGMP PDE5 inhibitors may also be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, e.g. as a carrier, diluent or solubiliser. Alpha-, beta- and gamma-cyclodextrins are most commonly used and suitable examples are described in WO-A-91/11172, WO-A-94/02518 and WO-A-98/55148.

Generally, in humans, oral administration of the cGMP PDE5 inhibitors is the preferred route, being the most convenient. In circumstances where the recipient suffers from a swallowing disorder or from impairment of drug absorption after oral administration, the drug may be administered parenterally, sublingually or buccally.

The cGMP PDE5 inhibitors can also be administered in combination with other active agents. Preferred agents include: compounds which modulate the action of a trial natriuretic factor (also known as atrial natriuretic peptide), such as inhibitors of neutral endopeptidase; compounds which inhibit angiotensin-converting enzyme such as enalapril, and combined inhibitors of angiotensin-converting enzyme and neutral endopeptidase such as omapatrilat; angiotensin receptor antagonists such as losartan; substrates for NO-synthase, i.e. L-arginine; calcium-channel blockers such as amlodipine; antagonists of endothelin receptors and inhibitors of endothelin-converting enzyme; cholesterol lowering agents e.g. statins and fibrates; antiplatelet and antithrombotic agents, e.g. tPA, uPA, warfarin, hirudin and other thrombin inhibitors, heparin, thromboplastin activating factor inhibitors; insulin sensitising agents such as rezulin and hypoglycaemic agents such as glipizide; L-DOPA and carbidopa; acetylcholinesterase inhibitors such as donezipil or steroidal; COX2 inhibitors; pregabalene; gabapentene; tricyclic antidepressants, e.g. amitriptiline; non-steroidal anti-inflammatory agents; and angiotensin-converting enzyme (ACE) inhibitors, e.g. quinapril. More preferred agents are: compounds which inhibit angiotensin-converting enzyme; angiotensin receptor antagonists; substrates for NO-synthase antagonists of endothelin receptors and inhibitors of endothelin-converting enzyme; cholesterol lowering agents; and insulin sensitising agents and hypoglycaemic agents. Especially, insulin sensitising agents and hypoglycaemic agents.

It is to be appreciated that all references herein to alleviating include curative, palliative and prophylactic treatment.

It is to be appreciated that all references herein to a patient suffering from spinal cord injury include mammals and are not intended to mean only the human.

### EXAMPLES

The following examples are illustrative only and are not intended to limit the scope of the present invention.

### EXAMPLE

Sildenafil citrate was administered to three male patients of ages 30-73, who have sexual dysfunction and somatic pain.

Patients:

Patient 1: Male, Age 37, 7^{th} thoracic vertebrae injury and complete paraplegia. He was constantly suffering from inferior limb and superior limb numbness and pain. Erectile dysfunction. Every day behavior: Wheel chair.

Patient 2: Male, Age 73, 12^{th} thoracic vertebrae injury and complete paraplegia. He was constantly suffering from inferior limb pain. Erectile dysfunction. Every day behavior: Wheel chair.

Patient 3: Male, Age 30, 6^{th} cervical vertebrae injury and incomplete quadriplegia. He was constantly suffering from limb pain and spasticity. Erectile dysfunction. Every day behavior: Wheel chair.

Patients 1 to 3 were dosed with a 50 mg tablet of VIAGRA® in the unit dosage form when feeling pain or spasticity.

Effect, Side Effect, Frequency of Dosage, etc.

Patient 1: Pain or spasticity in inferior limb and superior limb began to be alleviated 30 minutes after the administration, and the initial pain or spasticity was alleviated by about 50% one hour after the administration. Afterward, the effect of alleviating the pain or spasticity remained for around half day. Erectile dysfunction was improved. Light heat sensation was observed as a side effect, which disappeared in two to three hours. Frequency of dosage was three to four times a month, and the patient was dosed only when he felt severe pain or spasticity.

Patient 2: An effect of alleviating pain or spasticity emerged around one hour after the administration. Afterward, the effect of alleviating the pain or spasticity remained within continence for around one day. Erectile dysfunction was not improved. Thus, the patient was dosed with the tablet in the unit dosage form, for the purpose of only alleviating the pain or spasticity. No side effect was observed. Frequency of dosage was two to three times a month, and the patient was dosed only when he felt severe pain or spasticity.

Patient 3: An effect of alleviating pain or spasticity appeared around 45 minutes after the administration. Afterward, the effect of alleviating the pain or spasticity remained for around 5 to 6 hours. Erectile dysfunction was improved. No side effect was observed. Frequency of dosage was two to three times a month, and the patient was dosed only when he felt severe pain or spasticity or needed sexual intercourses.

Effects of sildenafil citrate on alleviating pain or spasticity in the patients suffering from spinal cord injury are summarised in the following Table 1.

TABLE 1
Effects of sildenafil citrate on alleviating pain or spasticity in the patients suffering from spinal cord injury

| | | | | | Effects of | | | |
|---|---|---|---|---|---|---|---|---|
| | | | | Emerging Time | alleviating | | | Improvement |
| | Type of Spinal | | | of Effect After | pain or | Duration of | | of Erectile |
| Subjects | Cord Injury | Age | Dose | Administration | spasticity | Effects | Side Effects | Dysfunction |
| Patient 1 | Th7—complete | 37 | 50 mg | 30 minutes | by about 50% | half day | Light heat | Yes |
| | paraplegia | | | | | | Sensation | |
| | | | | | | | (disappeared | |
| | | | | | | | in 2 to | |
| | | | | | | | 3 hours) | |
| Patient 2 | Th12—complete | 73 | 50 mg | one hour | by about 30% | about one day | No | No |
| | paraplegia | | | | | | | |
| Patient 3 | C6—incomplete | 30 | 50 mg | 45 minutes | by about 20% | 5-6 hours | No | No |
| | quadriplegia | | | | | | | |

As can be seen from the clinical test results as mentioned above, it was demonstrated that an administration of 50 mg sildenafil citrate alleviated the pain or spasticity by about 20 to 50% in 30 minutes to 1 hour after the administration, and such an effect remained for 5-6 hours to about one day.

It is considered that in about one third to one fourth of patients suffering from spinal cord injury, who also have pain or spasticity caused by the injury, such pain or spasticity could be alleviated by the administration of sildenafil citrate. In Patients 1 to 3 in the clinical tests, no effects of alleviating pain or spasticity could be found by analgesics other than the effective ingredients of the present invention, such as loxoprofen sodium (Loxonin®), diclofevac sodium (Voltaren®). Thus, it is expected that the effective ingredients of the present invention can be made use of as an agent for alleviating inferior pain or spasticity and/or reducing the customarily used analgesics, in addition to an agent for improving male erectile dysfunction, if they are used appropriately.

1. A method for alleviating pain or spasticity in a patient suffering from spinal cord injury, comprising the step of administering to the patient such an effective amount of a cGMP PDE5 inhibitor sufficient to alleviate the pain or spasticity.

2. The method according to claim 1, wherein the inhibitor is administered orally.

3. The method according to claim 1, wherein the daily dosage is 5 to 500 mg.

4. The method according to claim 1, wherein the inhibitor has an IC₅₀ at less than 100 nanomolar.

5. The method according to claim 1, wherein the inhibitor has a selectivity ratio in excess of 100.

6. The method according to claim 1, wherein the inhibitor is a compound of formula (I): 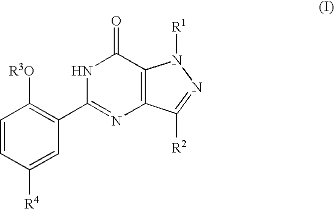
wherein R¹ is H; C₁-C₃ alkyl; C₁-C₃ perfluoroalkyl; or C₃-C₅ cycloalkyl;
R² is H; C₁-C₆ alkyl optionally substituted with C₃-C₆ cycloalkyl; C₁-C₃ perfluoroalkyl; or C₃-C₆ cycloalkyl;
R³ is C₁-C₆ alkyl optionally substituted with C₃-C₆ cycloalkyl; C₁-C₆ perfluoroalkyl; C₃-C₅ cycloalkyl; C₃-C₆ alkenyl; or C₃-C₆ alkynyl;
R⁴ is C₁-C₄ alkyl optionally substituted with OH, NR⁵R⁶, CN, CONR⁵R⁶ or CO₂R⁷; C₂-C₄ alkenyl optionally substituted with CN, CONR⁵R⁶ or CO₂R⁷; C₂-C₄ alkanoyl optionally substituted with NR⁵R⁶; (hydroxy)C₂-C₄ alkyl optionally substituted with NR⁵R⁶; (C₂-C₃ alkoxy)C₁-C₂ alkyl optionally substituted with OH or NR⁵R⁶; CONR⁵R⁶; CO₂R⁷; halo; NR⁵R⁶; NHSO₂NR⁵R⁶; NHSO₂R⁸; SO₂NR⁹R¹⁰; or phenyl, pyridyl, pyrimidinyl, imidazolyl, oxazolyl, thiazolyl, thienyl or triazolyl any of which is optionally substituted with methyl;
R⁵ and R⁶ are each independently H or C₁-C₄ alkyl, or together with the nitrogen atom to which they are attached form a pyrrolidinyl, piperidino, morpholino, 4-N(R¹¹)-piperazinyl or imidazolyl group wherein said group is optionally substituted with methyl or OH;
R⁷ is H or C₁-C₄ alkyl;
R⁸ is C₁-C₃ alkyl optionally substituted with NR⁵R⁶;
R⁹ and R¹⁰ together with the nitrogen atom to which they are attached form a pyrrolidinyl, piperidino, morpholino or 4-N(R¹²)-piperazinyl group wherein said group is optionally substituted with C₁-C₄ alkyl, C₁-C₃ alkoxy, NR¹³R¹⁴ or CONR¹³R¹⁴;
R¹¹ is H; C₁-C₃ alkyl optionally substituted with phenyl; (hydroxy)C₂-C₃ alkyl; or C₁-C₄ alkanoyl;
R¹² is H; C₁-C₆ alkyl; (C₁-C₃ alkoxy)C₂-C₆ alkyl; (hydroxy)C₂-C₆ alkyl; (R¹³R¹⁴N)C₂-C₆ alkyl; (R¹³R¹⁴NOC)C₁-C₆ alkyl; CONR¹³R¹⁴; CSNR R¹⁴; or C(NH)NR¹³R¹⁴;
and R¹³ and R¹⁴ are each independently H; C₁-C₄ alkyl; (C₁-C₃ alkoxy)C₂-C₄ alkyl; or (hydroxy)C₂-C₄ alkyl;
or a pharmaceutically acceptable salt thereof.

7. The method according to claim 1, wherein the inhibitor is sildenafil, or pharmaceutically acceptable salts thereof.

8. The method according to claim 1, wherein the daily dosage is 10 to 100 mg.
